(19) United States
(12) Patent Application Publication
(10) Pub. No.: US 2008/0159983 A1
(43) Pub. Date: Jul. 3, 2008

(54) Interleukin-15 antagonists for the treatment of anemia

(76) Inventors: Stephen Smiley, Saranac Lake, NY (US); Lawrence Johnson, Saranac Lake, NY (US); Isis Mullarky, Pembroke, VA (US)

Correspondence Address: KENYON & KENYON LLP, ONE BROADWAY, NEW YORK, NY, 10004, US

(21) Appl. No.: 12/005,767

(22) Filed: Dec. 27, 2007

Related U.S. Application Data
(60) Provisional application No. 60/878,399, filed on Dec. 29, 2006.

(51) Int. Cl. A61K 38/21; A61K 38/47; A61K 38/17; C12Q 1/00; C12Q 1/68; A61K 38/02; A61K 39/395

(52) U.S. Cl. 424/85.4; 424/94.61; 424/158.1; 514/12; 514/2; 435/4; 435/6

(57) ABSTRACT

The present invention relates to methods and compositions for the prevention and treatment of anemia. Anemia is characterized by a deficiency in red blood cells (RBCs) or hemoglobin, thereby reducing the supply of oxygen to tissues and organs. The invention is based on the discovery that interleukin-15 (IL-15) acts downstream of interferon-gamma (IFNγ) to suppress erythropoiesis. The methods and compositions of the invention may be used in the treatment of anemia accompanying a variety of illnesses characterized by a decrease in RBCs.

### SPECIFICATION

This research was supported by PHS grants HL72937, AI46571, AI61587, and AI49823. The United States Government may have rights in this invention.

### 1. INTRODUCTION

The present invention relates to methods and compositions for the prevention and treatment of anemia. Anemia is characterized by a deficiency in red blood cells (RBCs) or hemoglobin, thereby reducing the supply of oxygen to tissues and organs. The invention is based on the discovery that interleukin-15 (IL-15) acts downstream of interferon-gamma (IFNγ) to suppress erythropoiesis. The methods and compositions of the invention may be used in the treatment of anemia accompanying a variety of illnesses characterized by a decrease in RBCs.

### 2. BACKGROUND OF INVENTION

Anemia is a condition in which the blood becomes deficient in red blood cells (RBCs) or hemoglobin, thereby reducing the supply of oxygen to tissues and organs. Clinically significant anemia accompanies a variety of illnesses characterized by acute or chronic immune activation, including cancer, cardiac disease, autoimmunity, and infection (J. L. Spivak, J. L., Lancet 355:1707-1712 (2000); R. T. Means, Jr., Curr. Hematol. Rep. 2:116-121 (2003); G. Weiss and L. T. Goodnough, N. Engl. J. Med. 352:1011-1023 (2005). During life-threatening illnesses, anemia often correlates with an increased risk of early death (H. M. Muller, et al., Clin. Transplant. 15:343-348 (2001); S. Claster, J. Infect. Dis. 185 Suppl 2:S105-109 (2002); P. C. Lee et al., J. Am. Coll. Cardiol. 44:541-546 (2004); J. Vaglio et al., Am. J. Cardiol. 96:496-499 (2005).

Given these important clinical implications, the causes of anemia have been studied extensively. To maintain RBC homeostasis, the bone marrow continually produces new RBC through a process called erythropoiesis, while cells of the reticuloendothelial system phagocytose senescent RBCs and recycle iron, an essential component of oxygen-carrying hemoglobin. Anemia often results from both a decrease in erythropoiesis and an increase in the rate at which RBCs are lost from the circulation (Spivak, J. L. 2000 Lancet 355:1707-1712; Means, R. T., Jr. 2003 Curr. Hematol. Rep. 2:116-121; Weiss, G., and L. T. Goodnough. 2005. Anemia of chronic disease. N. Engl. J. Med. 352:1011-1023).

Studies of the mechanisms promoting anemia during genetic disorders, chronic disease, and infection have increasingly focused on roles for IFNγ (Means, R. T., Jr. 2003. Curr. Hematol. Rep. 2:116-121; Weiss, G., and L. T. Goodnough. 2005 N. Engl. J. Med. 352:1011-1023), an inflammatory cytokine that suppresses the growth of erythroid colony forming units (CFU-E) in vitro (Zoumbos, N. C., J. Y. Djeu, and N. S. Young. 1984 J. Immunol. 133:769-774; Means, R. T., Jr., S. B. Krantz, J. Luna, S. A. Marsters, and A. Ashkenazi. 1994 Blood 83:911-915). However, the specific pathways linking IFNγ to suppressed erythropoiesis in vivo remain poorly defined, thereby hampering development of palliative therapies.

### 3. SUMMARY OF THE INVENTION

The present invention provides methods and compositions useful in the treatment of anemia. The methods and compositions of the invention can be used in the treatment of anemia that accompanies a variety of illnesses characterized by a RBC deficiency. The invention is based on the discovery that IL-15 plays an essential role in mediating the IFNγ-induced suppression of erythropoiesis.

Accordingly, the present invention relates to a method of treating anemia through administration of compounds that inhibit, or reduce, IL-15 mediated signal transduction. Such compositions include, but are not limited to, peptides, including soluble peptides, small organic or inorganic molecules, therapeutic nucleic acid molecules, including antisense, ribozymes and siNA, all of which function as IL-15 inhibitors. Additionally, anti-IL-15 antibodies, anti-IL-15R antibodies, or fragments thereof, may be used to treat anemia.

Still further, the present invention relates to screening assays that utilize the IL-15 gene and/or IL-15 gene product for the identification of compounds which modulate IL-15 mediated signal transduction. In a preferred embodiment of the invention, the compound is one that is capable of inhibiting the activity of IL-15 and effectively reducing or inhibiting anemia. Such compounds can be used as agents to prevent and/or treat anemia. The invention further provides pharmaceutical compositions comprising a biologically active agent that modulates the activity of IL-15 in combination with a pharmaceutically acceptable carrier.

### 4. BRIEF DESCRIPTION OF THE FIGURES

FIG. 1. Kinetic analysis of anemia, reticulocyte production, and levels of IFNγ during acute T. gondii infection. Wild type C57BL/6 mice were infected with 10 ME49 cysts as described in materials and methods. (A) RBC numbers, (B) percentage of circulating reticulocytes, and (C) plasma IFNγ levels were evaluated on the indicated days. Asterisks depict significant differences between sham-infected control mice (open circles) and T. gondii-infected mice (closed circles; p<0.02). Data depict the mean and standard deviation of four mice per group.

FIG. 2. IL-12, IFNγ, and IFNγR are required for infection-stimulated suppression of erythropoiesis. Wild type (WT) C576BL/6 mice or mice deficient (KO) in IFNγ, IFNγR (γR), IL-12p35 (p35), or IL-12p40 (p40) were infected with 10 ME49 cysts and blood samples were collected 8 days later. (A) Percentage of circulating reticulocytes and (B) plasma IFNγ levels for sham-infected mice (open bars) and T. gondii-infected mice (closed bars). Asterisks in (A) depict significant differences between sham-infected and infected mice (p<0.001). Asterisks in (B) depict significant reductions when comparing WT infected mice and KO infected mice (p<0.001). Data depict the mean and standard deviation of at least five mice per group.

FIG. 3. Infection-stimulated suppression of erythropoiesis is T-bet independent, but STAT4, and STAT1-dependent. Wild type (WT) C576BL/6 mice or mice deficient (KO) in STAT4, STAT1, or T-bet were infected with 10 ME49 cysts and blood samples were collected 8 days later. (A) Percentages of circulating reticulocytes and (B) plasma IFNγ levels are shown for 14 sham-infected mice (open bars) and T. gondii-infected mice (closed bars). Asterisks in (A) depict significant differences between sham-infected and infected mice (p<0.008). Asterisks in (B) depict significant reductions when comparing WT infected mice and KO infected mice (p<0.0005). Data depict the mean and standard deviation of at least five mice per group.

FIG. 4. Infection-stimulated suppression of erythropoiesis is IL-15 dependent. Wild type (WT) C576BL/6 mice or mice deficient (KO) in IL-15 or IFNγ were infected with 10 ME49 cysts and samples were collected 8 days later. (A) Percentage of circulating reticulocytes (B) plasma IFNγ levels and (C) hepatic IL-15 mRNA levels are shown for sham-infected (open bars) and infected (closed bars) animals. Asterisks depict significant differences between the indicated comparisons (p<0.0001; n.s.=not significant). Data depict the mean and standard deviation of at least seven mice per group.

FIG. 5. Suppression of CFU-E growth by IFNγ is IL-15 dependent. Bone marrow cells derived from wild type (WT) or IL-15-deficient (KO) mice were cultured in triplicate as described in Materials and Methods. The indicated cytokines or antibodies were added to the medium at the initiation of culture. Asterisks depict significant differences between the indicated comparisons (** p<0.005; * p<0.03; n.s.=not significant). Data depict the average of data pooled from two independent experiments

### 5. DETAILED DESCRIPTION OF THE INVENTION

Described herein is the discovery that the cytokine IL-15 mediates IFNγ-induced suppression of red blood cell production, resulting in anemia. Thus, the present invention relates to methods and compositions for blocking, or reducing, IL-15 activity, thereby preventing IFNγ-induced suppression of red blood cell production. The methods and compositions of the invention may be used to treat disorders characterized at least in part by anemia.

### 5.1 Inhibition of IL-15 Activity

Described below are methods and compositions for treating anemia wherein the IL-15 gene, IL-15 gene product, and/or IL-15R is used as a therapeutic target. Such compositions include, but are not limited to, peptides, including soluble peptides, small organic or inorganic molecules, therapeutic nucleic acid molecules, including antisense, ribozymes and siNA, all of which function as IL-15 inhibitors. Additionally, anti-IL-15 antibodies, anti-IL-15R antibodies, or fragments thereof, may be used to treat anemia. Such antibodies and fragments thereof include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with antigens.

The outcome of a treatment is designed to produce in a treated subject a healthful benefit, which in the case of anemia, includes but is not limited to an increase in red blood cell count. As discussed, in detail below, successful treatment of anemia can be brought about by techniques which serve to decrease IL-15 mediated signal transduction. Activity can be decreased by, for example, directly decreasing IL-15 gene product activity and/or by decreasing the level of IL-15 gene expression and/or by inhibiting the activity of the IL-15R.

In a specific embodiment of the invention, anti-IL-15 antibodies or anti-IL-15R antibodies can be utilized to treat anemia. Such antibodies can be generated using standard techniques. Such antibodies include but are not limited to polyclonal, monoclonal, Fab fragments, single chain antibodies, chimeric antibodies, and the like.

Antibodies that specifically recognize one or more epitopes of IL-15, or epitopes of conserved variants of IL-15, or peptide fragments of IL-15 are encompassed by the invention. Antibodies that specifically recognize one or more epitopes of the IL-15R, or epitopes of conserved variants of the IL-15R, or peptide fragments of the IL-15R are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For production of antibodies, various host animals may be immunized by injection with an IL-15 protein or IL-15R. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (Bacille Calmette-Guerin) and Corynebacterium parvum.

Polyclonal antibodies comprising heterogeneous populations of antibody molecules, may be derived from the sera of the immunized animals. Monoclonal antibodies may be obtained by any technique which provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclasses thereof. The hybridoma producing the mAb of this invention may be cultivated in vitro or in vivo. Production of high titres of Mabs in vivo makes this the presently preferred method of production.

In addition, techniques developed for the production of “chimeric antibodies” by splicing the genes from a mouse antibody molecule of appropriate antigen specificity together with genes from a human antibody molecule of appropriate biological activity can be used (Morrison et al., 1984, Proc Nat'l. Acad. Sci., 81:6851-6855; Neuberger et al., 1984, Nature, 312: 604-608; Takeda et al. 1985, Nature 314: 452-454). Alternatively, techniques developed for the production of humanized antibodies (U.S. Pat. No. 5,585,089) or single chain antibodies (U.S. Pat. No. 4,946,778 Bird, 1988, Science 242: 423-426; Huston et al., 1988, Proc. Nat'l. Acad. Sci USA, 85: 5879-5883; and Ward et al., 1989, Nature 334: 544-546) may be used to produce antibodies that specifically recognize one or more epitopes of IL-15.

The aforementioned methods can also be used to prepare antibodies to the IL-15R or fragments or subunits thereof, and to IL-15, or fragments thereof, bound to the IL-15R, or subunits or fragments thereof.

In a specific embodiment of the invention, antibodies that may be used in the practice of the invention include, but are not limited to those described in Villadsen et al. (Journal of Clinical Investigation 112:1571-1580, 2003), the MiKβ1 monoclonal antibody (Morris et al., Proc. Natl. Acad. Sci USA 103:401-406, 2006) and humax-IL15 (Baslund et al., Arthritis Rheum Suppl S: 1706, 2003).

In yet another embodiment of the invention, the level of IL-15 expression can be modulated using IL-15 based oligonucleotide molecules including but not limited to antisense, ribozyme, or RNAi approaches to inhibit or prevent translation of IL-15 mRNA transcripts or triple helix approaches to inhibit transcription of the IL-15 gene (herein after referred to as “therapeutic nucleic acid molcules”). Antisense, RNAi and ribozyme approaches involve the design of oligonucleotides (either DNA or RNA) that are complementary to IL-15 mRNA. The antisense, siNA or ribozyme oligonuclotides will be targeted to complementary IL-15 mRNA transcripts and prevent translation. Absolute complementarity, although preferred, is not required. One skilled in the art can ascertain a tolerable degree of mismatch by use of standard procedures to determine the melting point of the hybridized complex. Mammalian IL-15 sequences that may be used in the design of antisense, RNAi and ribozymes include those disclosed in D. M. Anderson, et al., Genomics 25 (1995), pp. 701-706 and H. Krause, B. et al. Cytokine 8 (1996), pp. 667-674, which are incorporated by reference herein in their entirety.

In a preferred embodiment of the invention, double-stranded short interfering nucleic acid (siNA) molecules may be designed to inhibit IL-15 expression. In one embodiment, the invention features a double-stranded siNA molecule that down-regulates expression of the IL-15 gene product, wherein said siNA molecule comprises about 15 to about 28 base pairs.

In one embodiment, the invention features a double stranded short interfering nucleic acid (siNA) molecule that directs cleavage of a IL-15 RNA via RNA interference (RNAi), wherein the double stranded siNA molecule comprises a first and a second strand, each strand of the siNA molecule is about 18 to about 28 nucleotides in length, the first strand of the siNA molecule comprises nucleotide sequence having sufficient complementarity to the IL-15 RNA for the siNA molecule to direct cleavage of the IL-15 RNA via RNA interference, and the second strand of said siNA molecule comprises nucleotide sequence that is complementary to the first strand.

The use of antisense molecules as inhibitors of gene expression is a specific, genetically based therapeutic approach (for a review, see Stein, in Ch. 69, Section 5 “Cancer: Principle and Practice of Oncology”, 4th ed., ed. by DeVita et al., J. B. Lippincott, Philadelphia 1993). The present invention provides the therapeutic use of nucleic acids of at least six nucleotides that are antisense to the IL-15 gene or a portion thereof. An “antisense” IL-15 nucleic acid as used herein refers to a nucleic acid capable of hybridizing to a portion of a IL-15 RNA (preferably mRNA) by virtue of some sequence complementarity. The antisense molecules will bind to the complementary IL-15 gene mRNA transcripts and reduce or prevent translation.

In yet another embodiment of the invention, ribozyme molecules designed to catalytically cleave IL-15 mRNA transcripts can also be used to prevent translation of IL-15 m-RNA and expression of IL-15. (See, e.g., PCT International Publication WO90/11364, published Oct. 4, 1990; Sarver et al., 1990, Science 247:1222-1225). Ribozymes are enzymatic RNA molecules capable of catalyzing the specific cleavage of RNA (For a review see, for example Rossi, J., 1994, Current Biology 4:469-471). The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety.

Alternatively, endogenous IL-15 gene expression can be reduced by targeting deoxyribonucleotide sequences complementary to the regulatory region of the IL-15 gene (i.e., the IL-15 promoter and or enhancer) to form triple helical structures that prevent transcription of the IL-15 gene in targeted cells in the body. (See generally, Helene, C. et al., 1991, Anticancer Drug Des. 6:569-584 and Maher, L J, 1992, Bioassays 14:807-815).

Therapeutic nucleic acid molecules such as RNAi, antisense and ribozyme molecules which inhibit IL-15 gene expression can be used in accordance with the invention to reduce the level of IL-15 gene expression, thereby effectively reducing the level of IL-15 activity. Still further, triple helix molecules can be utilized in reducing the level of IL-15 gene activity.

Such therapeutic nucleic acid molecules, i.e., RNAi, antisense, ribozyme and triple helix forming oligonucleotides, may be synthesized using standard methods known in the art for the synthesis of DNA and RNA molecules. These include techniques for chemically synthesizing oligodeoxyribonucleotides and oligoribonucleotides, such as for example, solid phase phosphoramidite chemical synthesis. The nucleic acid molecule can be DNA or RNA or chimeric mixtures or derivatives or modified versions thereof, single-stranded or double-stranded. The nucleic acid molecule can be modified at the base moiety, sugar moiety, or phosphate backbone, for example, to improve stability of the molecule, hybridization, etc. The nucleic acid molecule may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the nucleic acid molecules may be conjugated to another molecule, e.g., a peptide, hybridization triggered cross-linking agent, transport agent, hybridization-triggered cleavage agent, etc.

Alternatively, the therapeutic nucleic acid molecules can be generated by in vitro and in vivo transcription of DNA sequences encoding the therapeutic nucleic acid molecules. Such DNA sequences can be incorporated into a wide variety of vectors which incorporate suitable RNA polymerase promoters such as the T7 or SP6 polymerase promoters.

Any technique which serves to selectively administer nucleic acid molecules to a cell population of interest can be used, for example, by using a delivery complex. Such a delivery complex can comprise an appropriate nucleic acid molecule and a targeting means. Such targeting means can comprise, for example, sterols, lipids, viruses or target cell specific binding agents. In a specific embodiment, pharmaceutical compositions comprising a therapeutic nucleic acid molecule are administered via biopolymers, liposomes, microparticles, or microcapsules. In various embodiments of the invention, it may be useful to use such compositions to achieve sustained release of the therapeutic nucleic acids. In a specific embodiment, it may be desirable to utilize liposomes targeted via antibodies to specific IL-15 expressing cells.

It is often difficult to achieve intracellular concentrations of the therapeutic nucleic acid molecule sufficient to suppress translation of endogenous mRNAs. Therefore, a preferred approach utilizes a recombinant DNA construct in which expression of the therapeutic nucleic acid molecule is placed under the control of a strong pol III or pol II promoter. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds.), 1994, Current Protocols in Human Genetics, John Wiley & Sons, NY.

The use of recombinant DNA constructs to transfect target cells in the patient will result in the transcription of sufficient amounts of the therapeutic nucleic acid molecule that will form complementary base pairs with the endogenous IL-15 gene transcripts and thereby prevent translation of the IL-15 gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA.

Such vectors can be constructed by recombinant DNA technology methods standard in the art. Vectors can be plasmid, viral, or others known in the art, used for replication and expression in mammalian cells. Expression of the sequence encoding a therapeutic nucleic acid can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct which can be introduced either directly into the tissue site, or via a delivery complex. Alternatively, viral vectors can be used which selectively infect the desired tissue.

In addition, soluble IL-15Rα chain may be used to inhibit the activity of IL-15 (Ruchatz et al., J Immunol 160:5654-5660 (1998). Alternatively, mutant IL-15 molecules may be utilized to inhibit IL-15 activity (Kim et al., J Immunology 160:5742-5748).

In yet another embodiment of invention, IL-15 activity may be inhibited through inhibition of the natural signal transduction pathway leading to induction of IL-15. For example, inhibition of IL-15 inducers, such as, for example, interferon γ and STAT1 and/or STAT4 may be used to reduce anemia.

In another embodiment of the invention, nucleic acid molecules comprising a sequence encoding a dominant negative mutant IL-15 protein or non-functional fragment or derivative thereof, are administered to inhibit IL-15 function by interfering with the interactions of IL-15 with the IL-15R. Specifically, the nucleic acid comprises a IL-15 nucleic acid that is part of an expression vector that expresses a dominant non-functional IL-15 protein or fragment or chimeric protein thereof. The function of IL-15 is thought to be mediated by IL-15-receptor interactions. Therefore, IL-15 mutants that are defective in function but effective in binding to its receptor can be used as a dominant negative mutant to compete with the wild type IL-15.

### 5.2. Screening Assays for Compounds that Modulate IL-15 Activity

The present invention further provides methods for the identification of compounds that may, through their interaction with the IL-15 gene or IL-15 gene product, affect the production of red blood cells.

The following assays are designed to identify compounds that modulate the activity of IL-15 gene (i.e., modulate the level of IL-15 gene expression and/or modulate the level of IL-15 activity). Compounds identified via assays such as those described herein may be useful for ameliorating the symptoms of anemia. It is to be noted that the compositions of the invention include pharmaceutical compositions comprising one or more of the compounds identified via such methods. Such pharmaceutical compositions can be formulated, for example, as discussed, below.

Assays may be utilized which identify compounds which bind to IL-15 gene regulatory sequences (e.g., promoter sequences) and which may modulate the level of IL-15 gene expression. Such methods for identifying compounds that modulate IL-15 gene expression, comprise, for example: (a) contacting a test compound with a cell or cell lysate containing a reporter gene operatively associated with a IL-15 gene regulatory element; and (b) detecting expression of the reporter gene product. IL-15 regulatory elements include those described in D. M. Anderson et al., Genomics 25 (1995), pp. 701-706 and H. Krause et al., Cytokine 8 (1996), pp. 667-674. Any reporter gene known in the art can be used, including but not limited to, green fluorescent protein, β-galactosidase, alkaline phosphatase, chloramphenicol acetyltransferase, etc.

Also provided is another method for identifying compounds that modulate IL-15 gene expression comprising: (a) contacting a test compound with a cell containing IL-15 transcripts; and (b) detecting the translation of the IL-15 transcript. The detection of IL-15 translation can be achieved using methods well known to those of skill in the art, including but not limited to immunoassays designed to detect the presence of IL-15 protein.

In yet another embodiment of the invention, in vitro systems may be designed to identify compounds capable of interacting with, e.g., binding to, the IL-15 gene product. Such compounds may be useful, for example, modulating the activity of the IL-15 gene product. Such compounds may function to disrupt normal IL-15/IL-15 receptor interactions.

The principle of the assays used to identify compounds that interact with the IL-15 gene product involves preparing a reaction mixture of the IL-15 gene product, or fragments thereof and the test compound under conditions and for a time sufficient to allow the two components to interact with, e.g., bind to, thus forming a complex, which can represent a transient complex, which can be removed and/or detected in the reaction mixture. These assays can be conducted in a variety of ways. For example, one method to conduct such an assay would involve anchoring L-15 gene product or the test substance onto a solid phase and detecting IL-15 gene product/test compound complexes anchored on the solid phase at the end of the reaction. In one embodiment of such a method, the IL-15 gene product or fragment thereof may be anchored onto a solid surface, and the test compound, which is not anchored, may be labeled, either directly or indirectly.

In yet another embodiment of the invention, in vitro systems may be designed to identify compounds capable of interacting with, e.g., binding to, the IL-15 receptor. Such compounds may be useful, for example, modulating the activity of the IL-15 receptor. Such compounds may function to disrupt normal IL-15/IL-15 receptor interactions.

The assays used to identify compounds that interact with the IL-15 receptor involve preparing a reaction mixture of the IL-15 receptor, or fragments thereof and the test compound under conditions and for a time sufficient to allow the two components to interact with, e.g., bind to, thus forming a complex, which can represent a transient complex, which can be removed and/or detected in the reaction mixture. These assays can be conducted in a variety of ways. For example, one method to conduct such an assay would involve anchoring IL-15 receptor or the test substance onto a solid phase and detecting IL-15 receptor/test compound complexes anchored on the solid phase at the end of the reaction. In one embodiment of such a method, the IL-15 receptor or fragment thereof may be anchored onto a solid surface, and the test compound, which is not anchored, may be labeled, either directly or indirectly.

In practice, microtitre plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for IL-15 gene product or the test compound to anchor any complexes formed in solution, and a labeled antibody specific for the other component of the possible complex to detect anchored complexes.

In accordance with the invention, a cell-based assay system can be used to screen for compounds that modulate the activity of IL-15 and/or IL-15R and thereby modulate the downstream events that result in suppression of erythropoiesis. To this end, cells that endogenously express IL-15R and that respond to IL-15 can be used to screen for such compounds. Such cells include, for example, bone marrow cells, T cells, B cells, NK cells, macrophages, thymic and bone-marrow cells, brain, intestine, liver, skeletal muscle, lung, heart and kidney. Alternatively, cell lines may be genetically engineered to express IL-15 and/or IL-15R and used for screening purposes. For screens utilizing host cells genetically engineered to express a functional IL-15R protein, it would be preferred to use host cells that are capable of responding to IL-15.

The present invention provides methods for identifying compounds that interfere, with IL-15 binding to IL-15R, or with downstream events caused by such binding i.e. IL-15 signal transduction and which inhibit red blood cell formation. Specifically, compounds may be identified that bind to IL-15R but do not activate it as IL-15 would, or that prevent assembly of the IL-15R heterotrimer, for example. Alternatively, compounds may be identified that modulate the activity of proteins that modify IL-15R, e.g. glycosylate, and thereby interfere with IL-15 signal transduction.

The present invention provides for methods for identifying a compound that inhibits IL-15 activity comprising (i) contacting a cell expressing IL-15R with a test compound in the presence of IL-15 and measuring the level of IL-15R activity; (ii) in a separate experiment, contacting a cell expressing IL-15R and IL-15 in the absence of a test compound, where the conditions are essentially the same as in part (i) and then (iii) comparing the level of IL-15R activity measured in part (i) with the level of IL-15R activity in part (ii), wherein a decrease level of IL-15R activity in the presence of the test compound indicates that the test compound is an IL-15 inhibitor.

In utilizing the cell systems described above, the cells expressing the IL-15R protein are exposed to a test compound or to vehicle controls e.g., placebos). After exposure, the cells can be assayed to measure the activity of IL-15 or the activity of the IL-15 dependent signal transduction pathway itself can be assayed.

The ability of a test molecule to modulate the activity of IL-15R may be measured using standard biochemical and physiological techniques. For example, activity of IL-15 may be assayed using a variety of different assays, including but not limited to, measuring tyrosine phosphorylation and nuclear translocation of STAT 3 or STAT5, phosphorylation of tyrosine kinases p₅₆^{lck} or p₇₂^{syk}, induction of bcl-2 expression, activation of fos/jun, stimulation of CTLL and dendritic cell proliferation activation of NK cells, protection from apoptosis, increased phagocytosis and bacterial clearance by monocytes. In practice, high throughput screens may be conducted using arrays of reactions. Such arrays may comprise at least one solid phase. Microtitre plates conveniently can be utilized as the solid phase.

Compounds which may be screened in accordance with the invention include, but are not limited to, small organic or inorganic compounds, peptides, antibodies and fragments thereof, and other organic compounds (e.g., peptidomimetics) that modulate IL-15 activity. Compounds may include, but are not limited to, peptides such as, for example, soluble peptides, including but not limited to members of random peptide libraries (see, e.g., Lam, K. S. et al., 1991, Nature 354:82-84; Houghten, R. et al., 1991, Nature 354:84-86); and combinatorial chemistry-derived molecular library made of D- and/or L-configuration amino acids, phosphopeptides including, but not limited to, members of random or partially degenerate, directed phosphopeptide libraries; (see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope binding fragments thereof), and small organic or inorganic molecules.

### 5.2.5 Treatment of Anemia

The condition of anemia is characterized by a lower than normal number of red blood cells (erythrocytes) in the blood, usually measured as a decrease in the amount of hemoglobin and red blood cells. Anemia may occur due to increased destruction of red blood cells, increased blood loss from the body, and inadequate production of red blood cells by the bone marrow, among others. In some instances anemia results from an inherited disorder, whereas in other instances the condition is caused by a nutritional problem, infection, or exposure to a drug or toxin.

Both hemoglobin (amount of hemoglobin in a set volume of blood) and hematocrit (percentage of red blood cells in a blood sample) values are used to define anemia. The normal range of hemoglobin values is 14 g/dL to 17.4 g/dL for adult men and 12.3 g/dL to 15.3 g/dL for nonpregnant women. The World Health Organization defines anemia as less than 12 g/dL for nonpregnant women and less than 13 g/dL for men. The normal average hematocrit for adult males is 46%, and the range is 40% to 52%. For adult females, the normal average hematocrit is 41% and the range is 35% to 47%. Values that fall below the lower limits can indicate anemia.

The present invention provides methods and compositions for treating anemia. Such anemias include those associated with chronic disease, malaria, cancer, HIV/AIDS, hepatitis C, critical illness, diabetes, inflammatory bowel disease, aging, kidney disease, and surgery.

Many medications increase the risk for anemia. Among these are certain antibiotics, some antiseizure medications (e.g., phenytoin), immunosuppressive drugs (e.g., methotrexate and azathioprine), antiarrhythmic agents (e.g., procainamide and quinidine), anticlotting drugs (e.g., aspirin, warfarin, and heparin), and cancer treatments (including drugs and radiation), among others. Certain types of anemia are caused by deficiency of certain factors in the body, including for example, B₁₂ deficiency, folate deficiency, iron deficiency, and glucose-6-phosphate dehydrogenase (G6PD) deficiency. The present invention provides for treatment of anemias induced by medications or factor deficiencies.

The compounds and nucleic acid sequences described herein can be administered to a patient at therapeutically effective doses to reduce or prevent anemia. A therapeutically effective dose refers to that amount of a compound sufficient to result in a healthful benefit in the treated subject.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized.

Pharmaceutical compositions for use in accordance with the present invention can be formulated in conventional manner using one or more physiologically acceptable carriers or excipients. Thus, the compounds and their physiologically acceptable salts and solvents can be formulated for administration by inhalation or insufflation (either through the mouth or the nose) or oral, buccal, parenteral or rectal administration. Compositions may be formulated into a form that allows for sustained release of the drug in the area to be treated.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably; formulated to give controlled release of the active compound. For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the inhibitor, preferably in purified form, together with a suitable amount of carrier so as to provide the form for proper administration to the patient. The formulation should suit the mode of administration.

As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art.

The compositions of the invention may be administered systemically. For such injections, the compositions may be in an injectible liquid suspension preparation or in a biocompatible medium which is injectible in liquid form and becomes semi-solid at the site of damaged tissue. A conventional syringe can be used.

The appropriate concentration of the composition of the invention which will be effective will depend on the nature of the disorder or condition, and can be determined by one of skill in the art using standard clinical techniques. The progress of the recipient receiving the treatment may be determined using assays that are designed to determine red blood cell count. The precise dose to be employed in the formulation will also depend on the route of administration, and the seriousness of the disease or disorder, and should be decided according to the judgment of the practitioner and each patient's circumstances. Effective doses may be extrapolated from dose response curves derived from in vitro or animal model test systems. Additionally, the administration of the compound could be combined with other known efficacious drugs if the in vitro and in vivo studies indicate a synergistic or additive therapeutic effect when administered in combination.

### 6. EXAMPLE

### Inteleukin-15 Modulated Erythropoiesis

### 6.1. Materials and Methods

Mice. Six- to ten-week old male mice were used for all experiments. C57BL/6 wild type mice were purchased from either Taconic (Germantown, N.Y.) or The Jackson Laboratory (Bar Harbor, Me.), or were bred at Trudeau Institute. Breeding stock of C57BL/6-backcrossed mice deficient in STAT1, STAT4, or T-bet were kindly provided by Christine Biron (Brown University), Mark Kaplan (Indiana University), and Laurie Glimcher (Harvard School of Public Health), respectively. C57BL/6-backcrossed mice deficient in IFNγ or the IFNγR were purchased from The Jackson Laboratory and C57BL/6-backcrossed IL-15-deficient mice were purchased from Taconic.

All animal experiments were reviewed and approved by the Trudeau Institute Animal Care and Use Committee. Mice at Trudeau Institute are free of known common viral pathogens of mice, as determined by periodic screening of sentinel animals by the University of Missouri Research Animal Diagnostic and Investigative Laboratory (Columbia, Mo.).

Infection. T. gondii strain ME49 was originally provided by Jack Remington (Palo Alto Medical Foundation, Palo Alto, Calif.) and has since been maintained by serial passage in C57BL/6 mice. For experimental infections, mice were infected perorally with 10 ME49 cysts in 0.1 ml diluted brain suspension obtained from chronically infected wild type mice, as previously described (Johnson, L. L., et al., 2003 J. Exp. Med. 197:801-806). Sham-infected mice received similarly diluted brain suspensions from uninfected animals.

Blood parameters. Blood samples were obtained by cardiac puncture of mice that were anticoagulated (500 units heparin, intravenously) a few minutes prior to euthanasia by carbon dioxide narcosis. Numbers of circulating RBC were determined by Coulter Counter (Beckman Coulter) after diluting blood 20-fold in 5 mM EDTA in PBS. To determine the percentage of circulating reticulocytes, 5 μl of blood was stained with 500 μl thiazole orange solution (Retic-Count, BD Biosciences) for 1 hr at room temperature, centrifuged at 800 g for 5 min, and resuspended in 1% formaldehyde (Van Hove, L., W. Goossens et al., 1990 Clin. Lab. Haematol. 12:287-299; Manodori, A. B., and F. A. Kuypers 2002 J. Lab. Clin. Med. 140:161-165). The percentage of thiazole orange-stained RBC (i.e. reticulocytes) was determined by flow cytometry, using forward and side scatter to gate on RBC. IFNγ protein levels in plasma were measured by sandwich ELISA (BD Biosciences).

Assay for CFU-E. Bone marrow cells were harvested from the tibia and fibia of 6-week old mice and cultured in MethoCult M3334 at 2×105 cells per 35 mm dish as per manufacturer's recommendations (StemCell Technologies, Vancouver, BC, Canada). At culture initiation, the medium was supplemented with murine IFNγ (100 ng/ml, Peprotech, Rocky Hill, N.J.), IL-15 (50-100 ng/ml, Peprotech), rabbit anti-murine IL-15 (10 μg/ml, Peprotech) and/or rabbit IgG control antibody (10 μg/ml, Peprotech). After 2 or 3 days of culture, total CFU-E per plate were enumerated with an inverted microscope. Statistics. Group means were compared using Student's t-test (Prism 4.0 GraphPad Software, Inc.).

### 6.2 Results

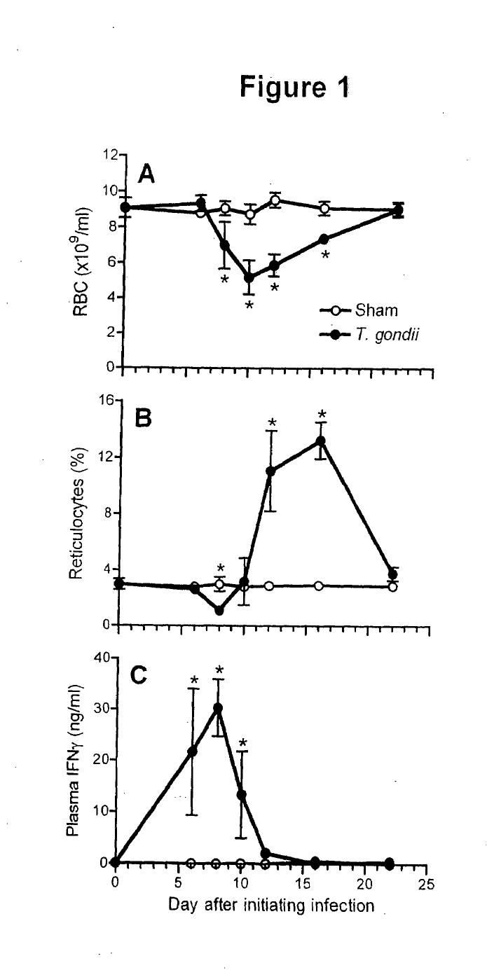

Decreased RBC production during acute toxoplasmosis. Peroral infection of wild type C57BL/6 mice with T. gondii transiently reduced the number of circulating RBC (FIG. 1A). This anemia became evident by day 8 after the initiation of T. gondii infection, peaked at day 10, and resolved by day 22. Consistent with anemias commonly associated with inflammation and chronic disease, the toxoplasmosis-associated anemia was normochromic and normocytic, as determined by the ADVIATM 120 Hematology System (Bayer Diagnostics, Tarrytown, N.Y.; not shown).

To assess whether suppressed erythropoiesis contributes to the toxoplasmosis-associated anemia, the production of RBC throughout the course of infection was quantified. Reticulocytes retain significant levels of RNA for 24-48 hours after their release from the bone marrow. As such, they can be identified and quantified using flow cytometric assays employing RNA binding dyes, such as thiazole orange (Van Hove, L., W. Goossens et al., 1990 Clin. Lab. Haematol. 12:287-299). It was observed that approximately 3% of circulating RBC in naïve mice stained positive with thiazole orange (FIG. 1B). Reticulocyte production remained at this basal level through day 6 after the initiation of T. gondii infection, but declined to 37% of basal levels on day 8 (FIG. 1B; p<0.001). Reticulocyte production rebounded by day 10 and achieved dramatically higher levels than those observed in sham-infected mice on days 12 and 18, before returning to baseline levels on day 22 (FIG. 1B). Thus, it was concluded that acute toxoplasmosis suppresses erythropoiesis.

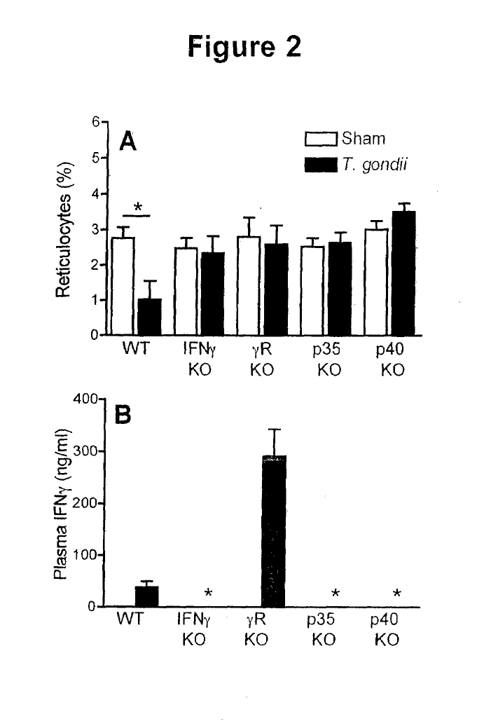

Roles for IFNγ, IFNγR and IL-12. IFNγ causes the anemia observed at day 8 after the initiation of T. gondii infection (Johnson, L. L., et al., 2003 J. Exp. Med. 197:801-806). Serum levels of IFNγ peak at that time (FIG. 1C). As shown in FIG. 2A, genetic deficiency in either IFNγ or the IFNγR prevented the decrease in reticulocyte production at day 8 after the initiation of T. gondii infection, despite increasing the parasite burden 1000-fold (Johnson, L. L., et al., 2003 J. Exp. Med. 197:801-806). It was concluded that IFNγ promotes anemia during acute toxoplasmosis, at least in part, by suppressing erythropoiesis.

IL-12 is a heterodimeric cytokine consisting of two subunits: p35 and p40 (11). It was observed that mice lacking the capacity to express either the IL-12p35 or p40 subunits, like IFNγ-deficient mice, did not exhibit decreased reticulocyte production at day 8 after the initiation of T. gondii infection (FIG. 2A). As reported by others (Gazzinelli, et al. 1994 J. Immunol. 153:2533-2543), robust production of IFNγ during acute toxoplasmosis required expression of IL-12 (FIG. 2B). Together, these data indicate that IL-12-stimulates production of IFNγ, which then signals through IFNγR to suppress erythropoiesis during acute toxoplasmosis.

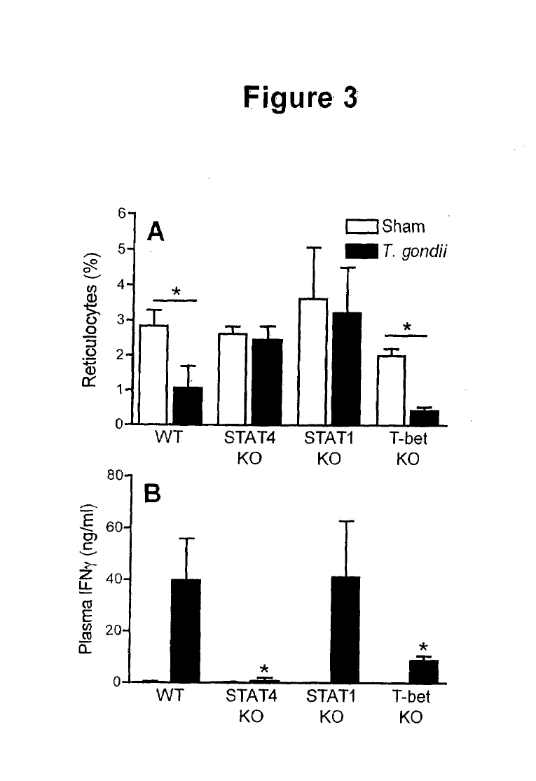

Suppression of erythropoiesis requires STAT4 and STAT1, but not T-bet. The transcription factors STAT4, STAT1, and T-bet each contribute to IFNγ production (Szabo, S. J. et al., 2003 Annu. Rev. Immunol. 21:713-758). It was observed that STAT4-deficient animals produced barely detectable levels of IFNγ at day 8 after the initiation of T. gondii infection (FIG. 3B). STAT4-deficiency also prevented the infection-stimulated decrease in reticulocyte production (FIG. 3A). In contrast, T-bet-deficiency only partially suppressed IFNγ production during acute toxoplasmosis (FIG. 3B) and failed to prevent the infection-stimulated decrease in reticulocyte production (FIG. 3A). Apparently, the residual IFNγ production in T-bet-deficient mice suffices to suppress erythropoiesis. Interestingly, and as recently reported (Gavrilescu, L. C. et al. 2004 Infect. Immun. 72:1257-1264), STAT1-deficiency did not significantly impact levels of IFNγ during acute toxoplasmosis (FIG. 3B). Nevertheless, STAT1-deficiency prevented the infectionstimulated decrease in reticulocyte production (FIG. 3A). It was concluded that the infection-stimulated, IFNγ-dependent, suppression of erythropoiesis is T-bet-independent, but STAT4- and STAT1-dependent. STAT4 apparently suppresses erythropoiesis by promoting IFNγ production whereas STAT1 presumably does so by acting downstream of IFNγ, as suggested by prior studies using distinct models (Pang, Q., et al. 2000 Mol. Cell. Biol. 20:4724-4735; Halupa, A., et al. 2005 Blood 105:552-561).

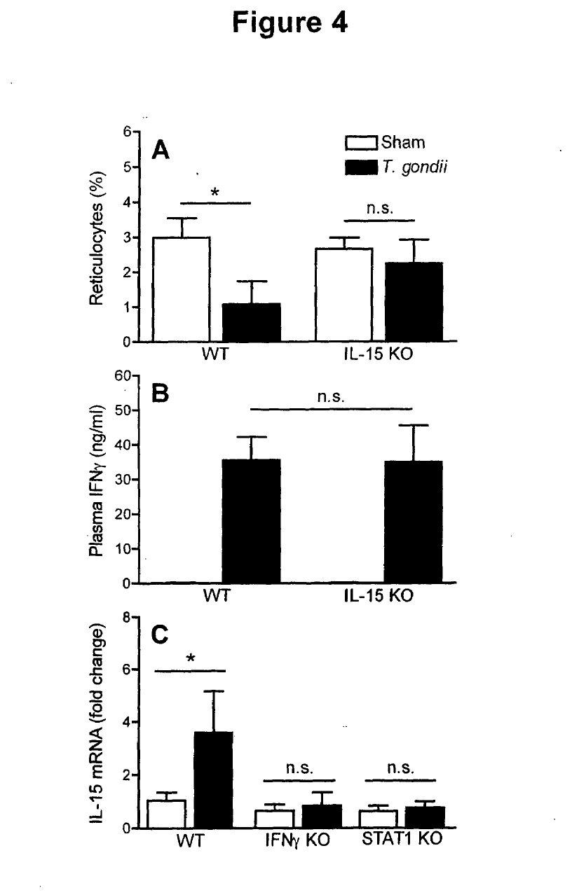

IL-15 suppresses erythropoiesis. A recent study reported that IL-15 is not a critical inducer of IFNγ during acute toxoplasmosis (Lieberman, L. A., et al. 2004 Infect. Immun. 72:6729-6732.). Similar levels of circulating IFNγ in wild type and IL-15-deficient mice were onserved at day 8 after the initiation of T. gondii infection (FIG. 4B). Parasite burden also was not significantly impacted by IL-15-deficiency (not shown). Nevertheless, IL-15-deficiency prevented the infection-stimulated decrease in reticulocyte production (FIG. 4A).

Prior studies found that IFNγ and STAT1 can participate in the induction of IL-15 expression in vitro (Musso, T., L. et al. 1999 Blood 93:3531-3539; Gysemans, C. A., et al. 2005 Diabetes 54:2396-2403; Wenxin, L., F. et al. 2005 Int. Immunol. 17:429-437). Acute T. gondii infection significantly increased hepatic expression of IL-15 mRNA in wild type mice (FIG. 4C). In striking contrast, acute toxoplasmosis failed to increase levels of hepatic IL-15 mRNA in mice deficient in IFNγ, IFNγR, or STAT1 expression (FIG. 4C). These observations indicate that IFNγ may suppress erythropoiesis by up-regulating STAT1-dependent expression of IL-15.

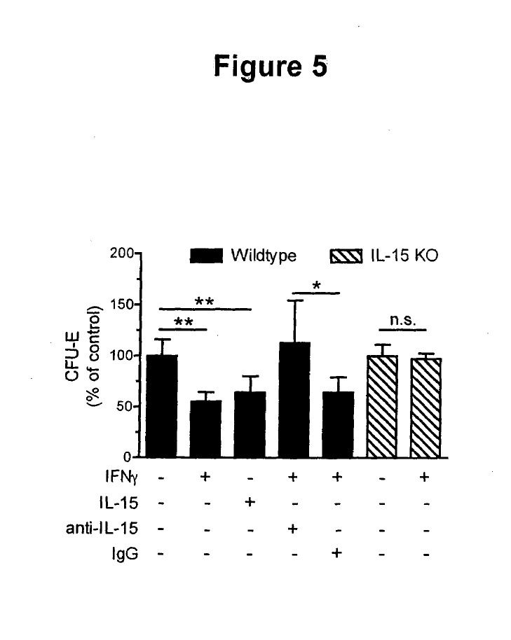

The relationships between IFNγ and IL-15, and their impacts on erythropoiesis in vitro was examined. Bone marrow cells from wild type mice were cultured in methylcellulose media supplemented with erythropoietin, which promotes the growth of erythroid progenitor cells. Consistent with prior reports (Zoumbos, N. C., J. Y. Djeu, and N. S. Young. 1984 J. Immunol. 133:769-774; Means, R. T. et al. 1994 Blood 83:911-915), supplementing the media with IFNγ suppressed the growth of CFU-E (FIG. 5). Remarkably, supplementing the media with IL-15 likewise suppressed the CFU-E growth (FIG. 5). To investigate whether IFNγ suppressed erythropoiesis via IL-15, a specific antibody was used to neutralize IL-15 activity in cultures supplemented with IFNγ. It was observed that addition of IL-15-specific antibody, but not control IgG, prevented IFNγ from suppressing the growth of CFU-E (FIG. 5). To further establish that IFNγ acts via IL-15, tests were conducted to determine whether IFNγ could suppress growth of CFU-E in cultures of IL-15-deficient bone marrow cells. It was found that IFNγ failed to suppress CFU-E growth in cell cultures derived from IL-15-deficient mice (FIG. 5). Taken together, these data establish that IFNγ suppresses the growth of CFU-E in vitro via impacts of IL-15.

Accumulating data indicates that IFNγ plays important roles in promoting numerous forms of anemia, in part by suppressing erythropoiesis. The data described herein demonstrates a critical role for IL-15 in the suppression of erythropoiesis by IFNγ. These findings indicate that IFNγ acts via STAT1 to induce IL-15, which then suppresses erythropoiesis. Notably, membrane-bound IL-15 is constitutively over-expressed by cultured bone marrow stromal cells derived from patients afflicted with aplastic anemia (Wenxin, L., et al. 2005 Int. Immunol. 17:429-437). The data indicates that therapeutic targeting of IL-15 and IL-15-dependent pathways will provide a therapeutic route to preventing IFNγ-mediated anemia while maintaining many protective attributes of this pleiotropic cytokine.

The present invention is not to be limited in scope by the specific embodiments described herein which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims. Throughout this application, various publications are referenced in parentheses. The disclosures of these publications in their entireties are hereby incorporated by reference into this application to more fully describe the state of the art to which this invention pertains.

1. A method of treating anemia comprising administration to an individual suffering from anemia an effective amount of an inhibitor of IL-15 activity.

2. The method of claim 1, wherein the inhibitor of IL-15 activity is an IL-15 antisense molecule.

3. The method of claim 1, wherein the inhibitor of IL-15 activity is an IL-15 ribozyme molecule.

4. The method of claim 1, wherein the inhibitor of IL-15 activity is an IL-15 siNA molecule.

5. The method of claim 1, wherein the inhibitor of IL-15 activity is an anti-IL-15 antibody.

6. The method of claim 1, wherein the inhibitor of IL-15 activity is a soluble IL-15Rα chain.

7. The method of claim 1, wherein the inhibitor is selected from the group consisting of interferon γ, STAT1, and STAT4.

8. The method of claim 1, wherein the inhibitor is a dominant negative mutant IL-15 protein.

9. A method of treating anemia comprising administration to an individual suffering from anemia an effective amount of an inhibitor of IL-15 mediated signal transduction.

10. The method of claim 9, wherein the inhibitor inhibits IL-15R activity.

11. The method of claim 10 wherein the inhibitor is an anti-IL-15R antibody.

12. A method for identification of an IL-15 inhibitor, useful for increasing the production of red blood cells, comprising (i) contacting a cell expressing IL-15R with a test compound in the presence of IL-15 and measuring the level of IL-15R activity; (ii) in a separate experiment, contacting a cell expressing IL-15R and IL-15 in the absence of a test compound, where the conditions are essentially the same as in part (i) and then (iii) comparing the level of IL-15R activity measured in part (i) with the level of IL-15R activity in part (ii), wherein a decrease level of IL-15R activity in the presence of the test compound indicates that the test compound is an IL-15 inhibitor.

13. A method for identifying a compound that inhibits IL-15 gene expression comprising: (a) contacting a test compound with a cell containing IL-15 transcripts; and (b) detecting the translation of the IL-15 transcript, wherein a decrease in the level of IL-15 translation in the presence of the test compound indicates that the test compound is an inhibitor of IL-15 gene expression.

14. A method for identifying a compound that modulates IL-15 gene expression, comprising: (a) contacting a test compound with a cell or cell lysate containing a reporter gene operatively associated with a IL-15 gene regulatory element; and (b) detecting expression of the reporter gene product wherein a decrease in the level of reporter gene product in the presence of the test compound indicates that the test compound is an inhibitor of IL-15 gene expression.

15. The method of claim 14, wherein the reporter gene product is selected from the group consisting of green fluorescent protein, β-galactosidase, alkaline phosphatase and chloramphenicol acetyltransferase.

16. The method of claim 12, 13 or 14, further comprising determining whether the test compound increases red blood cell production.
